(19) United States
(12) Patent Application Publication
(10) Pub. No.: US 2021/0174932 A1
(43) Pub. Date: Jun. 10, 2021

(54) INFORMATION PROCESSING DEVICE AND INFORMATION PROCESSING METHOD

(71) Applicant: SONY CORPORATION, TOKYO (JP)

(72) Inventor: YUKIKO ARAKAWA, TOKYO (JP)

(21) Appl. No.: 17/250,660

(22) PCT Filed: Jul. 16, 2019
(86) PCT No.: PCT/JP2019/027879

§ 371 Date: Feb. 17, 2021

(30) Foreign Application Priority Data
Aug. 30, 2018 (JP) 2018-161348

(51) Int. Cl. G16H 20/60; G16H 20/30; A61B 5/11

(52) U.S. Cl. CPC G16H 20/60; A61B 5/1112; G16H 20/30

(57) ABSTRACT

An information processing device (10) includes: a state change prediction unit (132) that predicts a change in a state of a user (U) due to actions scheduled by the user (U); and a notification information generation unit (134) that generates notification information for the user (U) on the basis of a prediction result obtained by the state change prediction unit (132).

### TECHNICAL FIELD

The present disclosure relates to an information processing device and an information processing method.

### BACKGROUND ART

In recent years, information processing devices having a function of predicting the future state of a user have become widespread. For example, the user can grasp his or her future health condition and work on improving lifestyle habits using the information processing device. PTL 1 discloses a method of predicting a user's future weight by obtaining a linear approximation line of the transition of the user's past weight data and correcting the linear approximation line on the basis of the user's gender, age, and fitness.

### CITATION LIST

### Patent Literature

[PTL 1]

JP 2015-014913A

### SUMMARY

### Technical Problem

However, the future state of the user will change depending on what the user will do in the future. In the method disclosed in PTL 1, it is difficult to accurately predict the future state of the user when the user performs an action that the user does not perform usually in the future.

Therefore, the present disclosure proposes new or improved information processing device and information processing method capable of predicting the user's future state more accurately.

### Solution to Problem

According to the present disclosure, there is provided an information processing device including: a state change prediction unit that predicts a change in a user state due to actions scheduled by the user; and a notification information generation unit that generates notification information for the user on the basis of a prediction result obtained by the state change prediction unit.

According to the present disclosure, there is provided an information processing method for causing a processor to execute: predicting a change in a user state due to actions scheduled by the user; and generating notification information for the user on the basis of the predicted change in the user state.

### Advantageous Effects of Invention

As described above, according to the present disclosure, it is possible to predict the user's future state more accurately.

Note that the effects described above are not necessarily limitative. With or in the place of the effects, there may be achieved any one of the effects described in this specification or other effects that may be grasped from this specification.

### BRIEF DESCRIPTION OF DRAWINGS

FIG. 1 is an explanatory diagram illustrating an overview of an information processing device according to a first embodiment of the present disclosure.

FIG. 2 is a block diagram illustrating an example of a configuration of the information processing device according to the embodiment.

FIG. 3 is a diagram illustrating an example of user's daily actions.

FIG. 4 is a diagram illustrating an example of user's scheduled actions.

FIG. 5 is a diagram illustrating an example of a classification table for classifying user's scheduled actions.

FIG. 6 is a diagram for describing an example of a method of calculating a user's basic calorie consumption.

FIG. 7 is a diagram for describing an example of a user's future calorie consumption.

FIG. 8 is a diagram illustrating an example of prediction results of a user's weight change.

FIG. 9 is a flowchart illustrating an example of the operation of the information processing device according to the embodiment.

FIG. 10 is an explanatory diagram illustrating an overview of an information processing device according to a second embodiment of the present disclosure.

FIG. 11 is a block diagram illustrating an example of a configuration of an information processing device according to the embodiment.

FIG. 12 is a flowchart illustrating an example of the operation of the information processing device according to the same embodiment.

FIG. 13 is a diagram illustrating an example of a prediction result of a user's weight change.

FIG. 14 is a diagram illustrating a hardware configuration example of the information processing device according to the embodiment of the present disclosure.

### DESCRIPTION OF EMBODIMENTS

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that description will be provided in the following order.

<1. First embodiment>

(1-1. Overview of information processing device)

(1-2. Configuration of information processing device)

(1-3. Operation of information processing device)

(1-4. Effects)

<2. Second embodiment>

(2-1. Overview of information processing device)

(2-2. Configuration of information processing device)

(2-3. Operation of information processing device)

(2-4. Effects)

<3. Modified example>

(3-1. First modified example)

(3-2. Second modified example)

<4. Hardware configuration>

<5. Conclusion>

### 1. FIRST EMBODIMENT

### 1-1. Overview of Information Processing Device

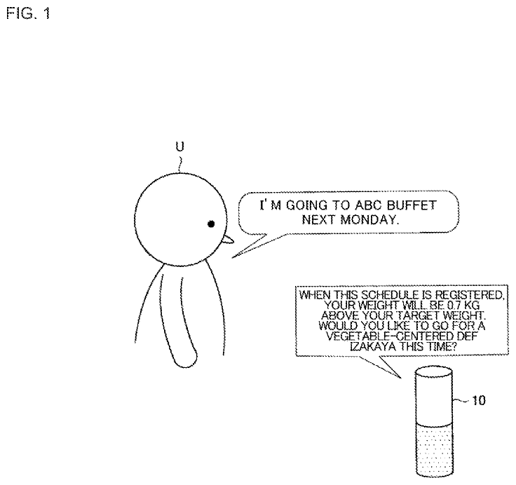

Hereinafter, an overview of an information processing device according to a first embodiment of the present disclosure will be described with reference to FIG. 1.

FIG. 1 is an explanatory diagram illustrating an overview of an information processing device according to the first embodiment of the present disclosure. As illustrated in FIG. 1, an information processing device 10 has a function of sending a notification or a proposal regarding the weight of a user U to the user U when an action scheduled by the user U (hereinafter, also simply referred to as a scheduled action) is input from the user U.

Specifically, when the scheduled action is input from the user U, the information processing device 10 predicts the change in weight occurring in the user U due to the scheduled action of the user U and sends a notification and a proposal regarding the body weight of the user U to the user U on the basis of the prediction result.

In the example illustrated in FIG. 1, when a scheduled action “go to ABC buffet next Monday” is input from the user U, the information processing device 10 predicts change in the body weight of the user U when the user U executes the scheduled action. Then, when the future body weight of the user U is predicted to exceed the target weight preset by the user U by 0.7 kg, the information processing device 10 notifies the user U that “when this schedule is registered, your weight will be 0.7 kg above your target weight.” Further, the information processing device 10 proposes to the user U, “Would you like to go for a vegetable-centered DEF izakaya this time?” to change the scheduled action so that the body weight of the user U does not exceed the target weight.

In the present disclosure, an example in which the information processing device 10 is an interactive agent that interacts with the user U will be described, but the information processing device 10 is not limited to such an example. For example, the information processing device 10 may be a smartphone, a mobile phone, a PHS (Personal Handyphone System), a portable game device, a robot, or the like.

### 1-2. Configuration of Information Processing Device

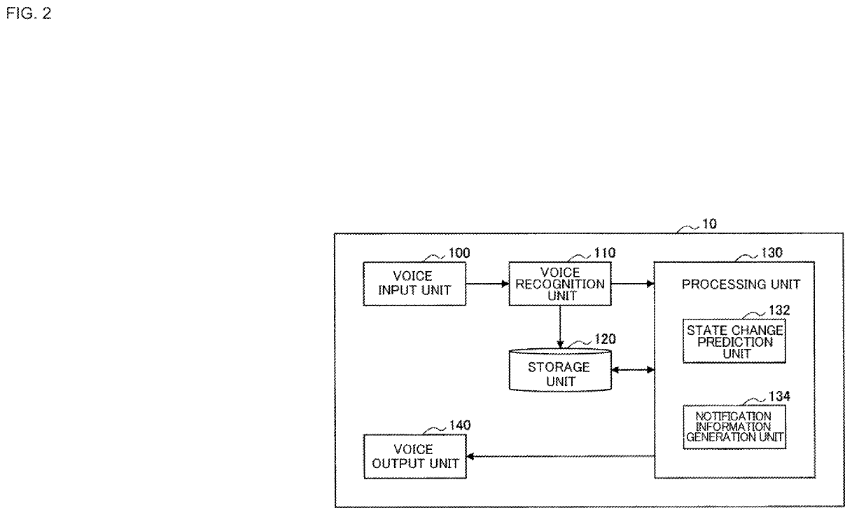

The overview of the information processing device 10 according to the present embodiment has been described above. Next, the configuration of the information processing device 10 according to the present embodiment will be described with reference to FIG. 2.

FIG. 2 is a block diagram illustrating an example of the configuration of the information processing device 10 according to the present embodiment. As illustrated in FIG. 2, the information processing device 10 includes a voice input unit 100, a voice recognition unit 110, a storage unit 120, a processing unit 130, and a voice output unit 140.

The voice input unit 100 has a function of collecting the voice of the user U. Further, the voice input unit 100 converts the collected sound to a voice signal and outputs the same to the voice recognition unit 110. Specifically, the voice input unit 100 is realized by a microphone, an amplifier, an A/D converter, and the like.

The voice recognition unit 110 has a function of acquiring the voice signal input from the voice input unit 100 and performing voice recognition. Specifically, the voice recognition unit 110 analyzes the voice signal on the basis of a known voice recognition technique, and recognizes the information included in the voice signal. For example, when the user U inputs a scheduled action to the information processing device 10, the voice recognition unit 110 recognizes the scheduled action information indicating the scheduled action of the user U included in the voice signal. Further, for example, when the user U inputs the body weight of the user U, the calories ingested by the user U, or the calories consumed by the user U to the information processing device 10, the voice recognition unit 110 recognizes weight information indicating the body weight of the user U, calorie intake information indicating the calorie intake of the user U, or calorie consumption information indicating the calorie consumption of the user U. The voice recognition unit 110 outputs the recognized information to the storage unit 120 and the processing unit 130.

The storage unit 120 has a function of storing the information input from the voice recognition unit 110 or the processing unit 130. For example, the storage unit 120 can store the above-mentioned scheduled action information, weight information, calorie intake information, calorie consumption information, and the like. The information stored in the storage unit 120 is not limited to these examples, and may include information regarding various settings made to the information processing device 10 by the user U.

The processing unit 130 has a function of processing the information input from the voice recognition unit 110 or the information stored in the storage unit 120. As illustrated in FIG. 2, the processing unit 130 includes a state change prediction unit 132 and a notification information generation unit 134.

The state change prediction unit 132 has a function of predicting a change in the state of the user U due to the scheduled action of the user U. For example, when a scheduled action is input from the user U, the state change prediction unit 132 calculates the calorie consumption of the user U on the day when the user U performs the scheduled action (hereinafter, also referred to as the future calorie consumption) and the calorie intake of the user U on the day when the user U performs the scheduled action (hereinafter, also referred to as the future calorie consumption) on the basis of the relationship between actions performed by user U in the past (hereinafter, also referred to as past actions), the calories consumed by user U on the day when user U performed past actions (hereinafter, also referred to as past calorie consumption), and the calories ingested by user U on the day when the user U performed past actions (hereinafter, also referred to as past calorie intake). Then, the state change prediction unit 132 predicts the future change in body weight of the user U on the basis of the future calorie consumption and the future calorie intake.

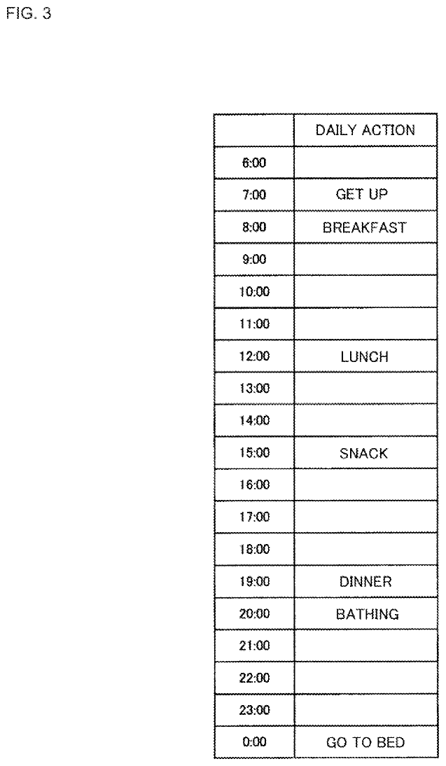

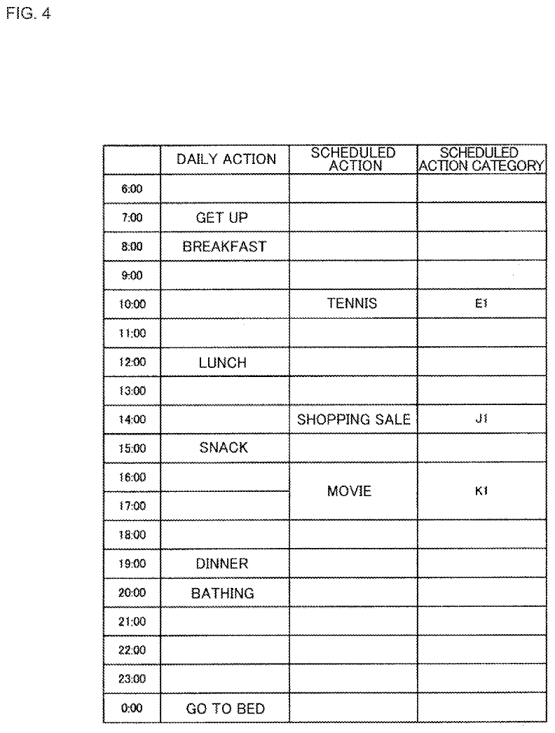

Hereinafter, an example of a method in which the state change prediction unit 132 predicts a change in the body weight of the user U will be described with reference to FIGS. 3 to 8. FIG. 3 is a diagram illustrating an example of daily actions of the user U registered in the information processing device 10 according to the present embodiment. FIG. 4 is a diagram illustrating an example of scheduled actions of the user U registered in the information processing device 10 according to the present embodiment.

As illustrated in FIG. 3, the daily actions of the user U are registered in the information processing device 10. Here, the daily actions mean actions performed by the user U on a daily basis. For example, when the user U takes a bath every day, the user U inputs to the information processing device 10 that he or she is taking a bath every day. The input by the user U is stored in the storage unit 120 via the voice input unit 100 and the voice recognition unit 110, whereby the daily action of taking a bath is registered in the information processing device 10. The means for registering the daily actions of the user U in the information processing device 10 is not limited to this example. For example, using a known automatic recognition technique, the information processing device 10 may automatically recognize the daily action of the user U and store the daily action of the user U in the storage unit 120.

When a scheduled action that is not registered in the daily action is newly registered in the information processing device 10, the state change prediction unit 132 classifies the scheduled action on the basis of the calorie intake that the user U ingests due to the scheduled action or the calorie consumption consumed by the user U due to the scheduled action. Here, the registration of the scheduled action in the information processing device 10 may be performed, for example, by the user U inputting the scheduled action to the information processing device 10, and is not limited to such an example. For example, using a known automatic recognition technique, the scheduled action of the user U may be automatically registered in the information processing device 10.

In the example illustrated in FIG. 4, scheduled actions such as “tennis”, “shopping sale”, and “movie” are newly registered for the daily actions of the user U. In such a case, the state change prediction unit 132 classifies the scheduled action of “tennis” to “E1”, the scheduled action of “shopping sale” to “J1”, and the scheduled action of “movie” to “K1”. Hereinafter, such a classification method will be described.

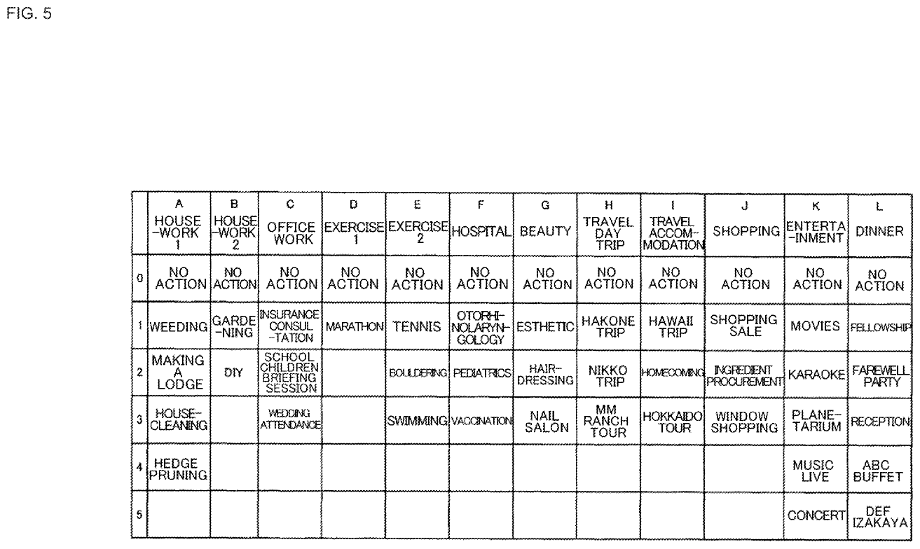

FIG. 5 is a diagram illustrating an example of a classification table for classifying the scheduled action of the user U to each category on the basis of the calorie intake or the calorie consumption. The classification table illustrated in FIG. 5 is defined in advance by the user U and registered in the information processing device 10. In the example illustrated in FIG. 5, each column from A to L defines a category illustrating actions with the same calorie intake or calorie consumption, and each row from 0 to 5 defines specific actions of the user U corresponding to each column. For example, column E defines a category called “exercise 2” that indicates exercise with moderate calorie consumption, and E1 to E3 define actions such as “tennis,” “bouldering,” and “swimming,” which are exercises with moderate calorie consumption. In addition, the action of “no action” is defined in E0, and when the user U does not perform the action corresponding to any one of E1 to E3, the action of the user U is classified to E0. When a scheduled action is newly registered, the state change prediction unit 132 classifies the registered scheduled action on the basis of the classification table, and stores the classified scheduled action in the storage unit 120.

When the user U finishes executing the scheduled action, the state change prediction unit 132 calculates the calorie consumption (past calorie consumption) consumed by the user U due to the scheduled action and the calorie intake (past calorie intake) ingested by the user U due to the scheduled action. Here, the past calorie intake is calculated as the total calorie value of food and drink ingested on the day when the user U performed the scheduled action. On the other hand, the past calorie consumption is calculated on the basis of the past calorie intake, the past weight gain or loss of the user U, and the basic calorie consumption of the user U. Here, the basic calorie consumption of the user U is a value indicating the calorie consumption of the user U on the day when the user U does not perform any action other than daily actions. Hereinafter, a method of calculating the past weight gain or loss of the user U and a method of calculating the basic calorie consumption will be described, and then a method of calculating the past calorie consumption will be described.

The method of calculating the past weight gain or loss of the user U and the method of calculating the basic calorie consumption will be described. The past weight gain or loss of the user U is calculated from the weight input daily from the user U to the information processing device 10. Specifically, the weight gain or loss is calculated by subtracting the body weight of the user U on the previous day from the body weight of the user U on a certain day.

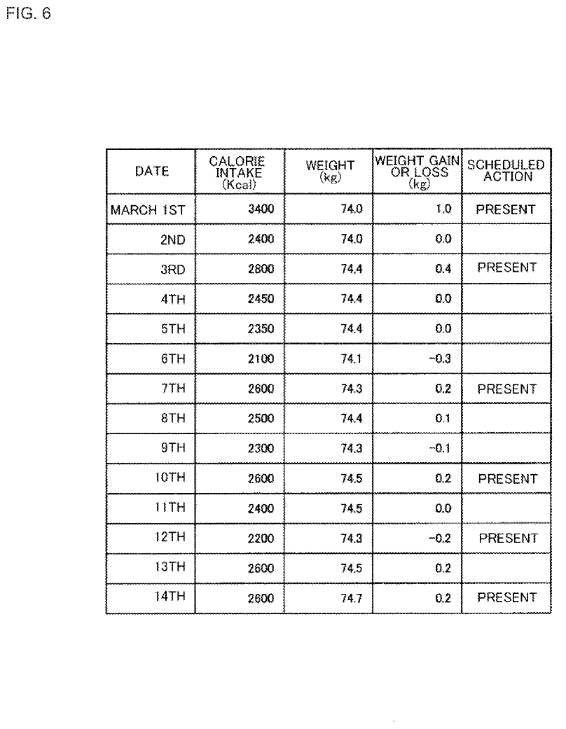

The basic calorie consumption of the user U is calculated as the average value of the calorie intake of the user U on the day when the weight gain or loss of the user U is equal to or less than a predetermined value among the days when the user U does not perform any action other than the daily actions (that is, the days when the scheduled action is not registered). The reason why limiting to the day when the weight gain or loss of the user U is equal to or less than the predetermined value is that it is highly likely that the scheduled action is not registered even though the user U is performing an action other than the daily actions on the day when the weight gain or loss is large. Hereinafter, an example of a method of calculating the basic calorie consumption of the user U will be described with reference to FIG. 6.

FIG. 6 is a diagram for describing an example of a method of calculating the basic calorie consumption of the user U. FIG. 6 illustrates an example of the past calorie intake, weight, weight gain or loss, and presence or absence of scheduled action of the user U. In description of the information processing device 10 according to the present embodiment, March 15 will be described as the present time in the following description. In other words, the days before March 14th will be described as the past and the days after March 16 as the future. In the example illustrated in FIG. 6, for example, when a predetermined value is 0.1 kg, the state change prediction unit 132 calculates 2400 kcal which is the average calorie intake on the days of March 2, 4, 5, 8, 9, and 11 when the scheduled action is not registered and the weight gain or loss is 0.1 kg or less as the basic calorie consumption of the user U. The predetermined value is not limited to such an example, and is appropriately set.

Next, an example of a method of calculating past calories consumption will be described. For example, the state change prediction unit 132 calculates the past calorie consumption of the user U on a certain past day on the basis of the calorie intake of the user U on that day, the weight gain or loss of the user U on that day, and the basic calorie consumption of the user U. Here, for example, in the information processing device 10, the relationship between the calorie intake and the weight gain or loss of the user U and the relationship between the calorie consumption and the weight gain or loss of the user U are registered. Specifically, in the information processing device 10, the fact that the body weight of the user U increases by 0.1 kg when the calorie intake of the user U increases by 100 kcal and the body weight of the user U decreases by 0.1 kg when the calorie consumption of the user U increases by 100 kcal is registered.

In such a case, for example, even if the user U ingested 200 kcal more calories than usual on a certain day, when there is no change in the body weight of the user U on that day, the state change prediction unit 132 can determine that the calorie consumption of the user U on that day is 200 kcal more than the basic calorie consumption and can calculate a value obtained by adding 200 kcal to the basic calorie consumption as the past calorie consumption of the user U on that day. Further, for example, even if the user U ingested the same calories as usual on a certain day, when the body weight of the user U on that day is decreased by 0.2 kg, the state change prediction unit 132 can determine that the calorie consumption of the user U on that day is 200 kcal more than the basic calorie consumption and can calculate a value obtained by adding 200 kcal to the basic calorie consumption as the past calorie consumption of the user U on that day. The state change prediction unit 132 stores the past calorie consumption and the past calorie intake calculated in this way in the storage unit 120.

In the description, an example in which the past calorie consumption is calculated and stored in the storage unit 120 has been illustrated, but the present disclosure is not limited to such an example. For example, the past calorie consumption acquired by a wearable terminal worn by the user U may be input to the information processing device 10 whereby the past calorie consumption is stored in the storage unit 120.

The state change prediction unit 132 calculates the future calorie consumption of the user U due to the scheduled action of the user U using the method for quantification theory, for example, on the basis of the relationship between the past calorie consumption and actions (hereinafter, also referred to as classified past actions) that have already been executed among the classified scheduled actions stored in the storage unit 120 as described above. Further, the state change prediction unit 132 calculates the future calorie intake of the user U due to the scheduled action of the user U using the method for quantification theory on the basis of the relationship between the classified past action and the past calorie intake. In the present embodiment, the state change prediction unit 132 calculates the future calorie consumption and the future calorie intake of the user U using the method of quantification theory type 1 known as one of the methods for quantification theory.

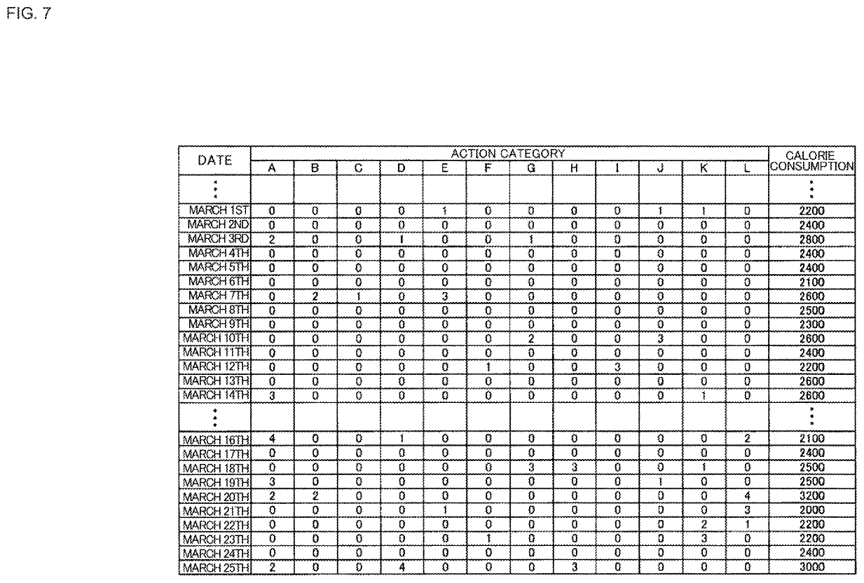

FIG. 7 is a diagram for describing an example of the future calorie consumption of the user U. In the example illustrated in FIG. 7, for days before March 14, the classified past actions and the past calories consumption are illustrated. Further, for days after March 16th, the classified scheduled actions and the future calorie consumption of the user U calculated by the state change prediction unit 132 using the method of quantification theory type 1 are illustrated. Although not illustrated here, the future calorie intake of the user U may be illustrated in the same manner as the future calorie consumption. The state change prediction unit 132 predicts a change in the body weight of the user U on the basis of the future calorie consumption of the user U and the future calorie intake of the user U.

For example, if the relationship between the calorie consumption and calorie intake of the user U and the body weight of the user U is known, the state change prediction unit 132 predicts the change in the body weight of the user U on the basis of the relationship. Specifically, when it is known that the body weight of the user U increases by 0.1 kg when the calorie (hereinafter, also referred to as a differential calorie) obtained by subtracting the calorie consumption of the user U on a certain day from the calorie intake of the user U on that day is 100 kcal, the state change prediction unit 132 can predict that the body weight of the user U on the day with a differential calorie of 100 kcal will increase by 0.1 kg.

In the above description, an example in which the state change prediction unit 132 calculates the future calorie consumption and the future calorie intake of the user U using the method of quantification theory type 1 has been illustrated, but the present disclosure is not limited to such an example. For example, the state change prediction unit 132 may calculate the future calorie consumption and the future calorie intake of the user U using another statistical analysis method instead of the method of quantification theory type 1.

For example, when the relationship between the past action, the past calorie consumption, and the past calorie intake of the user U is known, the state change prediction unit 132 may calculate the future calorie consumption and the future calorie intake of the user U on the basis of the relationship. Specifically, when it is known that the calorie consumption increases by 500 kcal when the user U plays tennis, the state change prediction unit 132 may calculate that the calorie consumption on the date when tennis is registered in the scheduled action will increase by 500 kcal.

The state change prediction unit 132 may not calculate the future calorie consumption and the future calorie intake of the user U when the number of pieces of data of the past action, the past calorie consumption, and the past calorie intake of the user U stored in the storage unit 120 is smaller than a predetermined number and it is determined that it is not possible to calculate the future calorie consumption and the future calorie intake of the user U. Here, the predetermined number may be a predetermined fixed value or a variable value appropriately determined by the state change prediction unit 132.

In the above description, an example in which the state change prediction unit 132 predicts the change in the body weight of the user U on the day when the scheduled action is registered has been illustrated, but the state change prediction unit 132 may predict changes in the body weight of the user U on days when the scheduled action is not registered.

As illustrated in FIG. 6, the body weight of the user U may change on the day when the scheduled action is not registered (for example, March 6). This is the case, for example, when the amount of breakfast of the user U is small and the calorie intake is less than usual. In this way, A0, B0, . . . , L0 in the classification table illustrated in FIG. 5 are stored as initial values in the storage unit 120 as the user U's actions on the day when no scheduled action of the user U is registered. In addition, when the user U finishes the action of the day, the state change prediction unit 132 calculates the calorie consumption consumed by the user U on that day and the calorie intake ingested by the user U on that day in the same manner as the above-mentioned past calories consumption and past calorie intake. Then, the state change prediction unit 132 stores the calculated calorie consumption and calorie intake in the storage unit 120 as the past calorie consumption and past calorie intake of the day.

As a result, the state change prediction unit 132 can calculate the calorie consumption and the calorie intake of the user U on a day when the user U has not registered any one scheduled action on the basis of the relationship between the classified past actions, the past calories consumption, and the past calorie intake stored in the storage unit 120. Then, the state change prediction unit 132 can predict the change in the body weight of the user U on the basis of the calculated calorie consumption and calorie intake.

In the above description, an example in which the state change prediction unit 132 predicts the change in the body weight of the user U due to the scheduled action of the user U on the basis of the relationship between the past action of the user U, the past calorie consumption, and the past calorie intake has been illustrated, but the present disclosure is not limited to this example. For example, the state change prediction unit 132 may predict the change in the body weight of the user U due to the scheduled action of the user U on the basis of the past action of the user U and the weight gain or loss of the user U on the day when the user U performed the past action instead of the relationship between the past action of the user U, the past calorie consumption, and the past calorie intake. Specifically, the state change prediction unit 132 can calculate the weight gain or loss of the user U due to the scheduled action of the user U using the method for quantification theory, for example, on the basis of the relationship between the classified past action and the weight gain or loss of the user U on the day when the user U performed the past action.

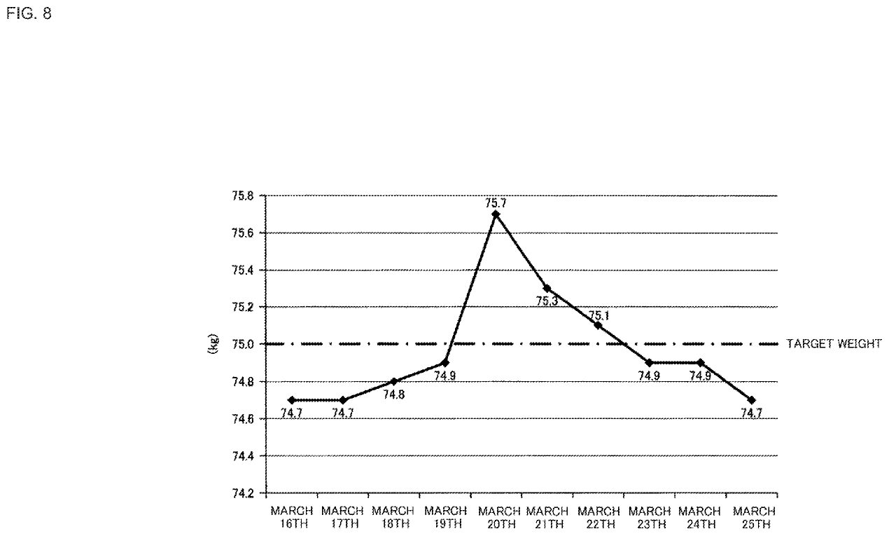

The notification information generation unit 134 has a function of generating notification information for the user U on the basis of the prediction result obtained by the state change prediction unit 132. Specifically, the notification information generation unit 134 generates notification information regarding the weight to the user U when the future body weight of the user U predicted by the state change prediction unit 132 exceeds a predetermined value preset by the user U. Hereinafter, an example in which the notification information generation unit 134 generates notification information for the user U will be described with reference to FIG. 8.

FIG. 8 is a diagram illustrating an example of the prediction result of the weight change of the user U. The horizontal axis illustrated in FIG. 8 indicates the future date (after March 16), and the vertical axis indicates the predicted body weight of the user U obtained by the state change prediction unit 132. Here, in the information processing device 10, 75.0 kg is set in advance by the user U as the target body weight of the user U. At this time, since the predicted body weight of the user U on March 20 is 75.7 kg, the notification information generation unit 134 determines that the target body weight of the user U is exceeded by 0.7 kg and generates the notification information for the user U.

Examples of the notification information generated by the notification information generation unit 134 include overweight notification information including notifying that the body weight of the user U exceeds the target weight, change proposal notification information including proposing to change the scheduled action of the user U, and additional proposal notification information including proposing the user U to add a new scheduled action. Here, the overweight notification information may specifically include a value indicating how much the predicted weight exceeds the target weight.

In the example illustrated in FIG. 1, the notification information generation unit 134 generates overweight notification information that “when this schedule is registered, your weight will be 0.7 kg above your target weight” and change proposal notification information that “would you like to go for a vegetable-centered DEF izakaya this time?”. On the other hand, the notification information generation unit 134 may generate additional proposal notification information that “Would you like to play tennis on Xth month and Yth day to suppress weight gain?” for example, in combination with or in place of the change proposal notification information.

The notification information generation unit 134 may generate notification information according to the setting by the user U, the past input, or the future state of the user U. For example, when the user U has low motivation for weight control and has set the information processing device 10 such that only the overweight notification information will be received as the notification information, the notification information generation unit 134 may generate only the overweight notification information.

For example, although the information processing device 10 sent a proposal to change the scheduled action of the user U in the past, when the response is rarely input from the user U or the proposal is often rejected, the notification information generation unit 134 may determine that the user U does not want the proposal to change the scheduled action and generate only the overweight notification information.

For example, when the future body weight of the user U exceeds the normal weight range of a general person of the same age, the notification information generation unit 134 may generate the notification information by changing an expression. Specifically, in the example illustrated in FIG. 1, the notification information generation unit 134 may generate notification information in a stronger expression that “please go for a vegetable-centered DEF izakaya this time?” instead of the change proposal notification information that “would you like to go for a vegetable-centered DEF izakaya this time?”.

The voice output unit 140 has a function of outputting the notification information generated by the notification information generation unit 134 to the user U. Specifically, the voice output unit 140 is realized by a speaker or the like.

### 1-3. Operation of Information Processing Device

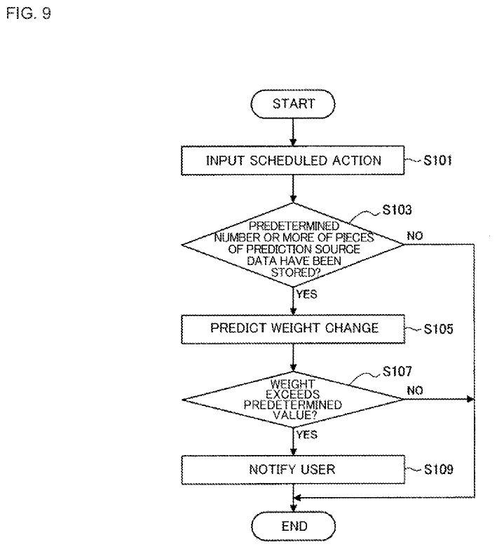

The configuration of the information processing device 10 according to the present embodiment has been described above. Next, the operation of the information processing device 10 according to the present embodiment will be described with reference to FIG. 9. FIG. 9 is a flowchart illustrating an example of the operation of the information processing device 10 according to the present embodiment.

First, when a scheduled action is input from the user U to the information processing device 10 (S101), the state change prediction unit 132 classifies the scheduled action to one of the actions in the classification table illustrated in FIG. 5.

Subsequently, the state change prediction unit 132 determines whether the number of pieces of data of the classified past action, past calorie consumption, and past calorie intake stored in the storage unit 120 is equal to or greater than a predetermined number (S103). When the number of pieces of data of the classified past action, past calorie consumption, and past calorie intake stored in the storage unit 120 is equal to or greater than the predetermined number (S103: Yes), the state change prediction unit 132 calculates the future calorie consumption and future calorie intake of the user U on the basis of the classified past action, past calories consumption, and past calories consumption stored in the storage unit 120. Subsequently, the state change prediction unit 132 predicts the future weight change of the user U on the basis of the calculated future calorie consumption and the future calorie intake of the user U (S105).

Subsequently, the notification information generation unit 134 determines whether the future body weight of the user U predicted by the state change prediction unit 132 exceeds a predetermined value preset by the user U (S107). When the predicted future body weight of the user U exceeds the predetermined value preset by the user U (S107: Yes), the notification information generation unit 134 generates notification information regarding the weight to the user U. Then, the voice output unit 140 outputs the notification information to the user U (S109).

### 1-4. Effects

The effects obtained by the information processing device 10 according to the present embodiment will be described. The information processing device 10 according to the present embodiment includes the state change prediction unit 132 that predicts a change in the body weight of the user U due to an action scheduled by the user U. As a result, the information processing device 10 can predict the future body weight of the user U with higher accuracy even when the user U performs an action that the user U does not perform usually in the future.

The state change prediction unit 132 predicts the change in the body weight of the user U due to the daily action of the user U in addition to the scheduled action of the user U. As a result, the information processing device 10 can predict the future body weight of the user U with higher accuracy regardless of the presence or absence of the action scheduled by the user U.

The information processing device 10 according to the present embodiment includes the notification information generation unit 134 that generates notification information regarding the weight for the user U when the future body weight of the user U predicted by the state change prediction unit 132 exceeds a predetermined value preset by the user U. In addition, the notification information includes at least one of overweight notification information, change proposal notification information, and additional proposal notification information. In this way, the user U can easily control the weight. Further, according to the information processing device 10, since the user U is specifically notified of how much the predicted weight exceeds the target weight, the user U can control the weight more positively.

The notification information generation unit 134 generates notification information according to the setting by the user U, the past input, or the future state of the user U. As a result, the user U can receive notifications according to the height of motivation of the user U for weight control and the future weight state of the user U.

### 2. SECOND EMBODIMENT

### 2-1. Overview of Information Processing Device

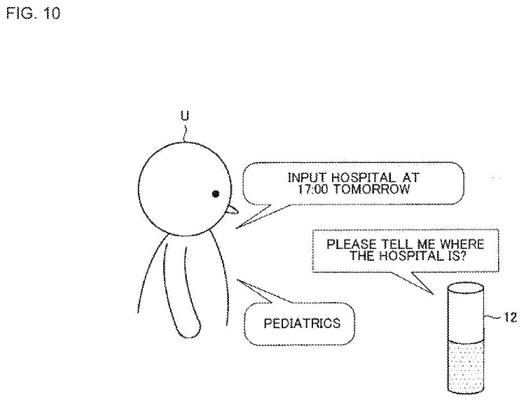

Next, the overview of an information processing device according to a second embodiment of the present disclosure will be described with reference to FIG. 10. FIG. 10 is an explanatory diagram illustrating an overview of the information processing device according to the second embodiment of the present disclosure. An information processing device 12 according to the second embodiment is different from the information processing device 10 according to the first embodiment in that, when the scheduled action input from the user U is not clear, the information processing device 12 inquires the user U about the details of the scheduled action. In the following description, basically, the content overlapping the description of the first embodiment will be omitted, and the differences from the first embodiment will be described.

Specifically, when the scheduled action is input from the user U, the information processing device 12 determines whether the scheduled action is clear. When it is determined that the scheduled action is not clear, the information processing device 12 inquires the user U about the details of the scheduled action. In the example illustrated in FIG. 10, when the scheduled action of “hospital at 17:00 tomorrow” is input from the user U, the information processing device 12 determines that the scheduled action is not clear and inquires about the details of the scheduled action such as “please tell me where the hospital is”. Then, the user U inputs a detailed scheduled action of “pediatrics”.

Since the functions and operations of the information processing device 12 after the detailed scheduled action is input from the user U are the same as those of the information processing device 10 according to the first embodiment described above, detailed description will be omitted here. Hereinafter, the configuration and operation of the information processing device 12 according to the present embodiment will be described in detail in order.

### 2-2. Configuration of Information Processing Device

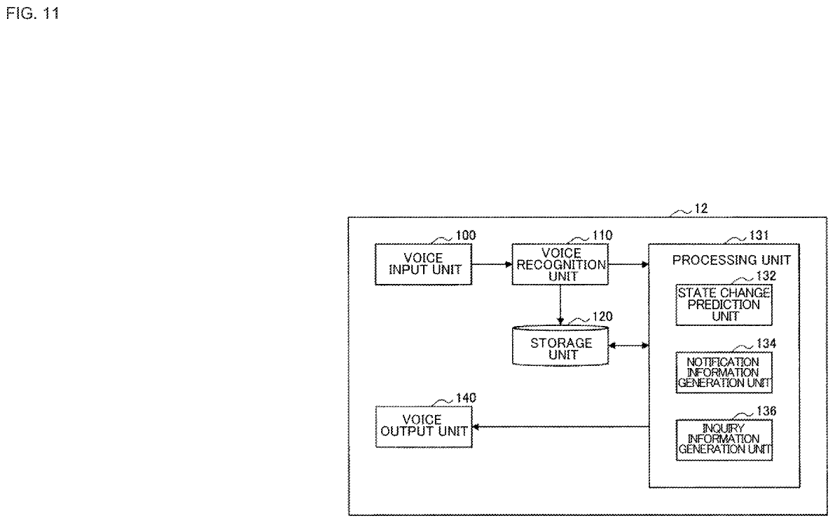

FIG. 11 is a block diagram illustrating an example of the configuration of the information processing device 12 according to the present embodiment. As illustrated in FIG. 11, the information processing device 12 includes a voice input unit 100, a voice recognition unit 110, a storage unit 120, a processing unit 131, and a voice output unit 140. Since the functions of the voice input unit 100, the voice recognition unit 110, the storage unit 120, and the voice output unit 140 are as described in the first embodiment, detailed description thereof will be omitted here.

The processing unit 131 has a function of processing the information input from the voice recognition unit 110 or the information stored in the storage unit 120. As illustrated in FIG. 11, the processing unit 131 includes a state change prediction unit 132, a notification information generation unit 134, and an inquiry information generation unit 136. Since the functions of the state change prediction unit 132 and the notification information generation unit 134 are as described in the first embodiment, detailed description thereof will be omitted here.

The inquiry information generation unit 136 has a function of generating inquiry information about the action scheduled by the user U for the user U. For example, the inquiry information generation unit 136 determines whether a scheduled action is clear when the scheduled action is input from the user U. Then, when the scheduled action is not clear, the inquiry information generation unit 136 generates inquiry information for inquiring the user U about the details of the scheduled action and outputs the inquiry information to the voice output unit 140.

Here, the inquiry information generation unit 136 can determine whether the scheduled action input from the user U is clear using the classification table illustrated in FIG. 5, for example. Specifically, the inquiry information generation unit 136 determines whether the scheduled action input from the user U corresponds to any of the categories in the classification table illustrated in FIG. 5. Then, the inquiry information generation unit 136 determines that the scheduled action input from the user U is not clear when the scheduled action input from the user U does not correspond to any of the categories in the classification table illustrated in FIG. 5.

Specifically, when the scheduled action of “hospital at 17:00 tomorrow” is input from the user U, the inquiry information generation unit 136 determines whether the action of “hospital” corresponds to any of the categories in the classification table illustrated in FIG. 5. Since the action of “hospital” is not present in the classification table illustrated in FIG. 5, the inquiry information generation unit 136 determines that the scheduled action is not clear.

Examples of the inquiry information generated by the inquiry information generation unit 136 include destination information for inquiring about the destination of the scheduled action of the user U, time information for inquiring the start time and end time of the scheduled action of the user U, and the like. Since the more the inquiry information, the more detailed scheduled action of the user U, the information processing device 12 can acquire, the future body weight of the user U can be predicted with higher accuracy.

The inquiry information generation unit 136 can generate inquiry information according to the setting by the user U or the past input by the user U. For example, if the user U does not want to receive a plurality of inquiries, the user U sets the information processing device 12 such that the user U will receive only one inquiry. In such a case, for example, the inquiry information generation unit 136 generates only the destination information as the inquiry information. Further, for example, although the information processing device 12 inquired about the start time and end time of the scheduled action of the user U in the past, when the response is rarely input from the user U, the inquiry information generation unit 136 determines that the user U does not want to be inquired about the start time and end time of the scheduled action and generates only destination information as inquiry information.

### 2-3. Operation of Information Processing Device

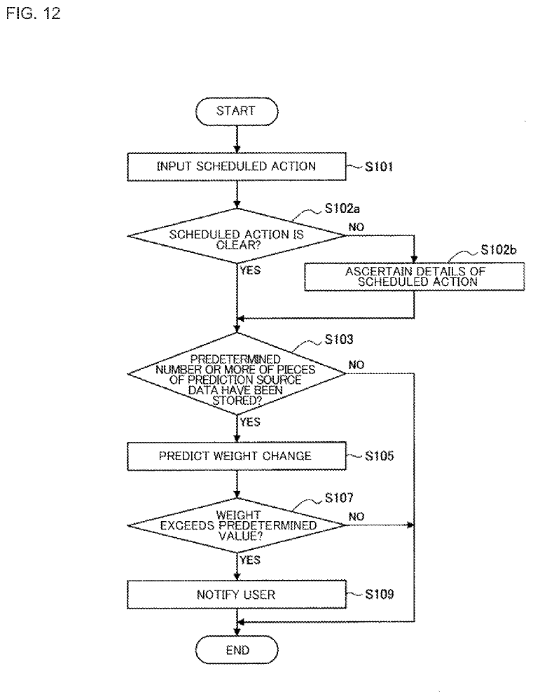

The configuration of the information processing device 12 according to the present embodiment has been described above. Next, the operation of the information processing device 12 according to the present embodiment will be described with reference to FIG. 12. FIG. 12 is a flowchart illustrating an example of the operation of the information processing device 12 according to the present embodiment.

In the example illustrated in FIG. 12, when the scheduled action is input from the user U to the information processing device 12 (S101), the inquiry information generation unit 136 determines whether the scheduled action of the user U is clear (S102a). When the scheduled action of the user U is not clear (S102a: No), the inquiry information generation unit 136 generates inquiry information for inquiring the user U about the details of the scheduled action. Subsequently, the voice output unit 140 ascertains the details of the scheduled action by outputting the inquiry information to the user U (S102b). Since the operation of the information processing device 12 after the detailed scheduled action is input from the user U is as described in the first embodiment, detailed description will be omitted here.

### 2-4. Effects

The effects obtained by the information processing device 12 according to the present embodiment will be described. The information processing device 12 according to the present embodiment includes the inquiry information generation unit 136 that generates inquiry information about an action scheduled by the user U for the user U. As a result, the information processing device 12 according to the present embodiment can acquire the details of the scheduled action of the user U even when the scheduled action input from the user U is not clear, and can predict the future body weight of the user U with higher accuracy.

### 3. MODIFIED EXAMPLE

Hereinafter, a modified example of the information processing device according to the embodiment of the present disclosure will be described. The modified examples described below may be applied alone to the embodiments of the present disclosure, or may be applied in combination to the embodiments of the present disclosure. Further, the modified example may be applied in place of the configuration described in the embodiments of the present disclosure, or may be additionally applied to the configuration described in the embodiments of the present disclosure.

### 3-1. First Modified Example

In the first embodiment described above, an example in which the notification information generation unit 134 generates notification information for the user U when the future body weight of the user U exceeds a predetermined value preset by the user U has been described. In the first modified example, the notification information generation unit 134 generates the notification information for the user U when the body weight of the user U at a future time point set in advance by the user U exceeds a predetermined value set by the user U. Here, the future time point indicates any one of a future date, time, or date and time.

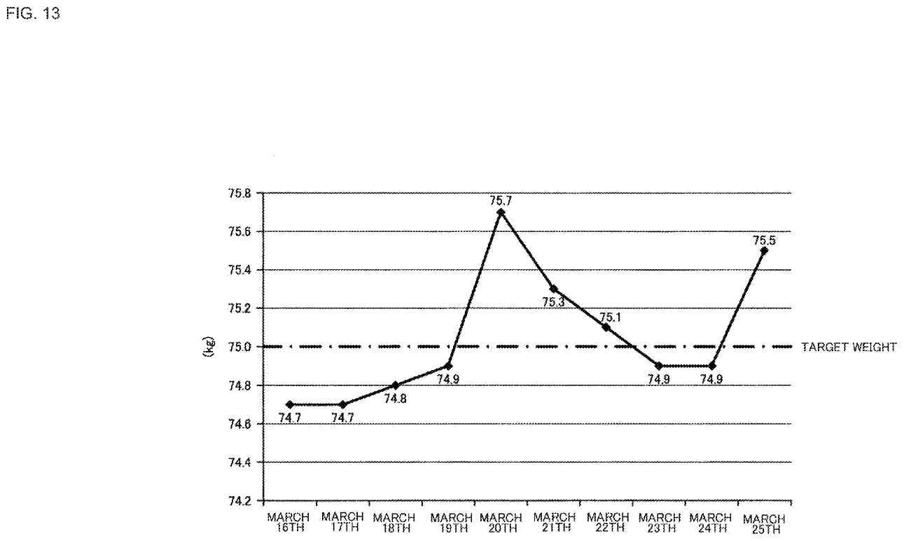

Hereinafter, an example of the operation of the notification information generation unit 134 in the first modified example will be described with reference to FIGS. 8 and 13. FIG. 13 is a diagram illustrating an example of the prediction result of the weight change of the user U similarly to FIG. 8. In the first modified example, in the information processing device 10, 75.0 kg is set in advance as the target body weight of the user U as of March 25. That is, the notification information generation unit 134 generates notification information for the user U when the body weight of the user U as of March 25 exceeds 75.0 kg, and does not generate the notification information for the user U when the body weight of the user U as of March 25 does not exceed 75.0 kg.

In the example illustrated in FIG. 8, although the predicted body weight of the user U on March 20 is 75.7 kg, which exceeds the target body weight of the user U, since the predicted body weight of the user U as of March 25 is 74.7 kg and does not exceed the target body weight of the user U, the notification information generation unit 134 does not generate the notification information for the user U. On the other hand, in the example illustrated in FIG. 13, since the predicted body weight of the user U as of March 25 is 75.5 kg, which exceeds the target body weight of the user U, the notification information generation unit 134 generates the notification information for the user U.

As described above, in the first modified example, the notification information generation unit 134 generates the notification information for the user U when the future body weight of the user U at the time point preset by the user U exceeds a predetermined value preset by the user U. As a result, the user U can manage the body weight of the user U at a preset time point.

### 3-2. Second Modified Example

In the second embodiment described above, an example in which the inquiry information generation unit 136 generates inquiry information about the scheduled action of the user U has been described. In the second modified example, the inquiry information generation unit 136 generates inquiry information about the daily action of the user U in addition to the scheduled action of the user U.

Hereinafter, an example of the operation of the inquiry information generation unit 136 in the second modified example will be described. First, the inquiry information generation unit 136 compares the body weight of the user U predicted by the state change prediction unit 132 with the actual body weight of the user U on the same date ex post facto. When the difference between the predicted body weight of the user U and the actual body weight of the user U is larger than a predetermined value, the inquiry information generation unit 136 determines that there is a difference between the scheduled action or the daily action originally scheduled by the user U on the date and the scheduled action or daily action performed actually.

In such a case, the inquiry information generation unit 136 generates inquiry information about the scheduled action or the daily action for the user U. For example, when it is known that there is often a difference in the action of user U having lunch, which is the daily action, the inquiry information generation unit generates inquiry information for the user U that “Is today's lunch the same as usual?” and outputs the inquiry information to the voice output unit 140. When the user U inputs a response to the inquiry output from the voice output unit 140, the information processing device 12 can acquire the action of the user U more accurately.

As described above, in the second modified example, the inquiry information generation unit 136 generates inquiry information about the daily action of the user U in addition to the scheduled action of the user U. As a result, the information processing device 12 can acquire the action of the user U more accurately and can predict the future body weight of the user U with higher accuracy.

### 4. HARDWARE CONFIGURATION

The embodiments of the present disclosure have been described above. Information processing such as prediction of weight change and generation of notification information described above is realized by cooperation of software and hardware of an information processing device described below.

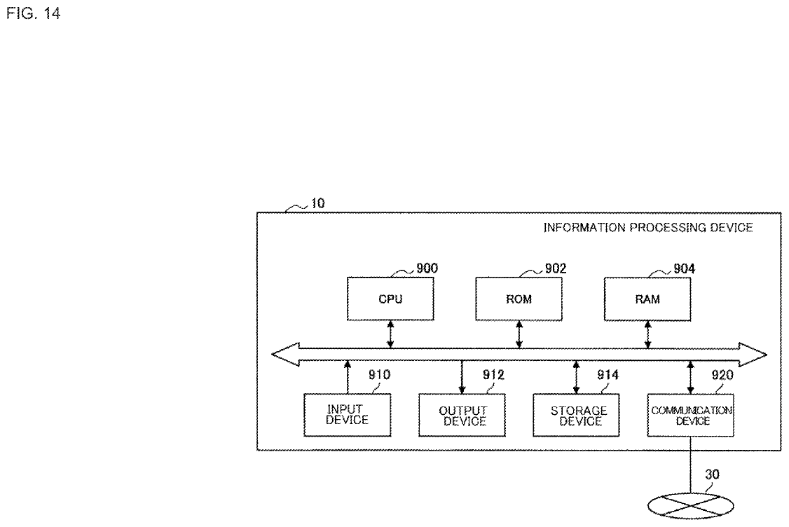

FIG. 14 is a diagram illustrating a hardware configuration of the information processing device. As illustrated in FIG. 14, the information processing device includes a CPU (Central Processing Unit) 900, a ROM (Read Only Memory) 902, a RAM (Random Access Memory) 904, an input device 910, an output device 912, a storage device 914, and a communication device 920.

The CPU 900 functions as an arithmetic processing device and a control device, and controls the overall operation in the information processing device according to various programs. Further, the CPU 900 may be a microprocessor. The ROM 902 stores programs, calculation parameters, and the like used by the CPU 900. The RAM 904 temporarily stores a program used in the execution of the CPU 900 and parameters and the like that change appropriately in the execution. These components are connected to each other by a host bus configured as a CPU bus or the like. By the cooperation of the CPU 900, ROM 902, and RAM 904 with the software, the functions of the state change prediction unit 132, the notification information generation unit 134, and the like are realized.

The input device 910 includes input means for the user to input information such as a mouse, a keyboard, a touch panel, buttons, a microphone, a switch, and a lever, and an input control circuit that generates an input signal on the basis of the input by the user and outputs the input signal to the CPU 900. By operating the input device 910, the user of the information processing device can input various kinds of data to the information processing device and instruct the information processing device to execute a processing operation.

The output device 912 includes a display device such as, for example, a liquid crystal display (LCD) device and an OLED (Organic Light Emitting Diode) device. Further, the output device 912 includes a voice output device such as a speaker and headphones. For example, the display device displays a captured image, a generated image, or the like. On the other hand, the voice output device converts voice data and the like to sound and outputs the sound.

The storage device 914 is a device for storing various types of data. The storage device 914 may include a storage medium, a recording device for recording data on the storage medium, a reading device for reading data from the storage medium, a deletion device for deleting data recorded on the storage medium and the like. As the storage device 914, for example, a semiconductor storage device, an optical storage device, a magnetic storage device such as a hard disk drive (HDD), an optical magnetic storage device, or the like is used.

The communication device 920 is, for example, a communication interface configured as a communication device or the like for connecting to the network 30. The communication device 920 may be a wireless LAN (Local Area Network) compatible communication device, an LTE (Long Term Evolution) compatible communication device, or a wire communication device that performs wired communication.

The network 30 is a wired or wireless transmission path for information transmitted from a device connected to the network 30. For example, the network 30 may include a public circuit network such as the Internet, a telephone circuit network, or a satellite communication network, various LANs (Local Area Networks) including Ethernet (registered trademark), a WAN (Wide Area Network) and the like. In addition, the network 30 may include a dedicated circuit network such as an IP-VPN (Internet Protocol-Virtual Private Network).

### 5. CONCLUSION

As described above, according to the embodiments of the present disclosure, it is possible to predict the future state of the user with higher accuracy.

While the preferred embodiments of the present disclosure have been described above with reference to the accompanying drawings, the technical scope of the present disclosure is not limited to the examples. A person skilled in the art may find various alterations and modifications within the scope of the technical idea described in the claims, and it should be understood that they will naturally come under the technical scope of the present disclosure.

For example, the steps of the above-described embodiments may not necessarily be executed chronically in the order described in the flowcharts. For example, the steps of the processing of the above-described embodiments may be processed in the order different from the order described in the flowcharts, or may also be processed in parallel.

Further, some of the functions of the information processing device described above may be implemented in a cloud server connected to the information processing device via the network 30. For example, the cloud server may have functions corresponding to the voice recognition unit 110, the storage unit 120, the state change prediction unit 132, and the notification information generation unit 134. In such a case, the information processing device can transmit the voice signal to the cloud server, and the cloud server can predict the change in the user state and generate the notification information for the user. Further, the information processing device can output the notification information received from the cloud server to the user.

In the above-described embodiments, an example in which the information processing device predicts the future body weight of the user as the future state of the user has been described, but the present disclosure is not limited to such an example. The present disclosure can be applied to the user's future abdominal circumference, body fat, BMI (Body Mass Index) as the user's future state as long as it is possible to predict the future state of the user on the basis of the relationship between the actions performed by the user in the past and the influential factors that influenced the change in the user state of the user when the action was performed.

Further, the effects described in this specification are merely illustrative or exemplified effects, and are not limitative. That is, with or in the place of the effects, the technology according to the present disclosure may achieve other effects that are clear to those skilled in the art based on the description of this specification.

Further, it is possible to create a computer program for causing the hardware such as the CPU, ROM, and RAM included in the information processing device to perform the same functions as each component of the information processing device described above. It is also possible to provide a storage medium having the computer program stored therein.

Note that, the following configurations also fall within the technical scope of the present disclosure.

(1)

An information processing device including:

a state change prediction unit that predicts a change in a user state due to actions scheduled by the user; and

a notification information generation unit that generates notification information for the user on the basis of a prediction result obtained by the state change prediction unit.

(2)

The information processing device according to (1), wherein the state change prediction unit predicts the change in the user state on the basis of a relationship between an action performed by the user in the past and an influential factor that influences the change in the user state when the action was performed.

(3)

The information processing device according to (1) or (2), wherein the state change prediction unit predicts the change in the user state due to daily actions of the user in addition to the action scheduled by the user.

(4)

The information processing device according to any one of (1) to (3), wherein the user state includes the body weight of the user.

(5)

The information processing device according to any one of (2) to (4), wherein the influential factor includes at least one of a calorie consumption or a calorie intake of the user.

(6)

The information processing device according to any one of (1) to (5), wherein the notification information generation unit generates the notification information when the prediction result exceeds a predetermined value set by the user.

(7)

The information processing device according to (6), wherein the notification information generation unit generates the notification information when the prediction result exceeds the predetermined value at a time point set in advance by the user.

(8)

The information processing device according to any one of (1) to (7), wherein the notification information generation unit generates the notification information according to a setting by the user, a past input, or a future state of the user.

(9)

The information processing device according to any one of (1) to (8), wherein the notification information includes a proposal to change the action scheduled by the user.

(10)

The information processing device according to any one of (1) to (9), further including an inquiry information generation unit that generates inquiry information about the action scheduled by the user for the user.

(11)

The information processing device according to (10), wherein the inquiry information generation unit generates inquiry information about daily actions of the user in addition to the action scheduled by the user.

(12)

The information processing device according to (10) or (11), wherein the inquiry information generation unit generates the inquiry information according to a setting by the user or a past input.

(13)

The information processing device according to any one of (1) to (12), wherein the information processing device is an interactive agent that interacts with the user.

(14)

An information processing method for causing a processor to execute:

predicting a change in a user state due to actions scheduled by the user; and

generating notification information for the user on the basis of the predicted change in the user state.

### REFERENCE SIGNS LIST

10, 12 Information processing device
100 Voice input unit
110 Voice recognition unit
120 Storage unit
130, 131 Processing unit
132 State change prediction unit
134 Notification information generation unit
136 Inquiry information generation unit
140 Voice output unit
U User

1. An information processing device comprising:
a state change prediction unit that predicts a change in a user state due to actions scheduled by the user; and
a notification information generation unit that generates notification information for the user on the basis of a prediction result obtained by the state change prediction unit.

2. The information processing device according to claim 1, wherein the state change prediction unit predicts the change in the user state on the basis of a relationship between an action performed by the user in the past and an influential factor that influences the change in the user state when the action was performed.

3. The information processing device according to claim 1, wherein the state change prediction unit predicts the change in the user state due to daily actions of the user in addition to the action scheduled by the user.

4. The information processing device according to claim 1, wherein the user state includes the body weight of the user.

5. The information processing device according to claim 2, wherein the influential factor includes at least one of a calorie consumption or a calorie intake of the user.

6. The information processing device according to claim 1, wherein the notification information generation unit generates the notification information when the prediction result exceeds a predetermined value set by the user.

7. The information processing device according to claim 6, wherein the notification information generation unit generates the notification information when the prediction result exceeds the predetermined value at a time point set in advance by the user.

8. The information processing device according to claim 1, wherein the notification information generation unit generates the notification information according to a setting by the user, a past input, or a future state of the user.

9. The information processing device according to claim 1, wherein the notification information includes a proposal to change the action scheduled by the user.

10. The information processing device according to claim 1, further comprising an inquiry information generation unit that generates inquiry information about the action scheduled by the user for the user.

11. The information processing device according to claim 10, wherein the inquiry information generation unit generates inquiry information about daily actions of the user in addition to the action scheduled by the user.

12. The information processing device according to claim 10, wherein the inquiry information generation unit generates the inquiry information according to a setting by the user or a past input.

13. The information processing device according to claim 1, wherein the information processing device is an interactive agent that interacts with the user.

14. An information processing method for causing a processor to execute:
predicting a change in a user state due to actions scheduled by the user; and
generating notification information for the user on the basis of the predicted change in the user state.
